(19) United States
(12) Patent Application Publication
(10) Pub. No.: US 2025/0319259 A1
(43) Pub. Date: Oct. 16, 2025

(54) Two-Part Stopper and Process for Making Same

(71) Applicant: Becton, Dickinson and Company, Franklin Lakes, NJ (US)

(72) Inventors: Alfred W. Prais, Hewitt, NJ (US); Glenn S. Posey, Sumter, SC (US); Leandro Forciniti, Wharton, NJ (US)

(21) Appl. No.: 18/635,482

(22) Filed: Apr. 15, 2024

(51) Int. Cl. A61M 5/315; B29C 45/00; B29K 23/00; B29K 25/00; B29L 31/00; B29L 31/26

(52) U.S. Cl. CPC A61M 5/31513; B29C 45/0001; A61M 2205/0216; A61M 2207/00; B29K 2023/10; B29K 2025/04; B29K 2995/007; B29K 2995/0077; B29L 2031/26; B29L 2031/7544

(57) ABSTRACT

The invention relates, in general, to various elastomeric, polymeric, and/or rubber formulations that can be used to form two-part, or dual-formulation, stopper designs where at least a portion of the stopper is formed using a low temperature processing step while the remainder of the stopper is formed using a high temperature processing step, methods of making same, and uses for such stoppers.

### BACKGROUND OF THE INVENTION

### Field of the Invention

The invention relates, in general, to various elastomeric, polymeric, and/or rubber formulations that can be used to form two-part, or dual-formulation, stopper designs where at least a portion of the stopper is formed using a low temperature processing step while the remainder of the stopper is formed using a high temperature processing step, methods of making same, and uses for such stoppers. In one embodiment, the present invention relates to a two-part stopper design formed from a stopper cap (or the distal or front portion) formed from a first elastomeric, polymeric, and/or rubber formulation that can be processed at a low temperatures (e.g., less than 130° C.), and a stopper body formed from a second elastomeric, polymeric, and/or rubber formulation that can be processed at a higher temperature (e.g., more than 150° C., or more than 175° C.) than the processing temperature used to produce the stopper cap. In one instance, the first elastomeric, polymeric, and/or rubber formulation is formed from one or more suitable elastomers, polymers, and/or rubbers such as one or more propylene-containing polymers (e.g., ethylene propylene diene monomer rubbers) or one or more crosslinked backbone unsaturated styrene terpolymers and are used to form at least the stopper cap (or the distal or front portion) of a stopper where such a compound is optionally laminated with one or more low processing temperature elastomeric, polymeric, and/or rubber lamination formulations (e.g., one or more suitable polyethylene such as a UHMWPE, copolymers thereof, and combinations thereof). In another instance, the second elastomeric, polymeric, and/or rubber formulation is formed from any one or more suitable elastomers, polymers, and/or rubbers having a processing temperature of at least 150° C., or at least 175° C., or even at least 200° C.

### Description of the Related Art

Various rubber compositions, compounded rubbers, and/or rubber compounds (hereinafter referred to just as “rubber compositions”) are known and used to form one-part closures for containers such as prefilled syringes and other medical applications. However, to date such rubber compositions suffer from one or more disadvantages including, but not limited to, high vulcanization, or even processing, temperatures (generally above 175° C., or even above 200° C.), require silicone oil lubrication, require lamination with a fluoropolymer-based barrier film (e.g., ETFE, PVDF, PTFE, etc.), require high break-out forces to function properly, and/or do not glide easily, or readily, over a wide range of surface interfaces with various glasses and/or plastics.

As such, there is a need in the art for a stopper design that permits a two-part designed to be realized where the stopper cap (or the distal or front portion) is formed from a first elastomeric, polymeric, and/or rubber formulation that can be processed at a low temperatures (e.g., less than 130° C.), and a stopper body formed from a second elastomeric, polymeric, and/or rubber formulation that can be processed at a higher temperature (e.g., more than 150° C., or more than 175° C.) than the processing temperature used to produce the stopper cap, where such stopper cap is optionally laminated with one or more low processing temperature elastomeric, polymeric, and/or rubber lamination formulations (e.g., one or more suitable polyethylene such as a UHMWPE, copolymers thereof, and combinations thereof). Additionally, in some embodiments, the stopper designs of the present invention do not require the use of a silicone oil lubrication, are able to operate properly at lower break-out forces, and/or glide easily, or readily, over a wide range of surface interfaces with various glasses and/or plastics.

Furthermore, there is a need in the art for a method to make such two-part stopper designs.

### SUMMARY OF THE INVENTION

The invention relates, in general, to various elastomeric, polymeric, and/or rubber formulations that can be used to form two-part, or dual-formulation, stopper designs where at least a portion of the stopper is formed using a low temperature processing step while the remainder of the stopper is formed using a high temperature processing step, methods of making same, and uses for such stoppers. In one embodiment, the present invention relates to a two-part stopper design formed from a stopper cap (or the distal or front portion) formed from a first elastomeric, polymeric, and/or rubber formulation that can be processed at a low temperatures (e.g., less than 130° C.), and a stopper body formed from a second elastomeric, polymeric, and/or rubber formulation that can be processed at a higher temperature (e.g., more than 150° C., or more than 175° C.) than the processing temperature used to produce the stopper cap. In one instance, the first elastomeric, polymeric, and/or rubber formulation is formed from one or more suitable elastomers, polymers, and/or rubbers such as one or more ethylene propylene-containing polymers (e.g., ethylene propylene diene monomer rubbers) or one or more crosslinked backbone unsaturated styrene terpolymers and are used to form at least the stopper cap (or the distal or front portion) of a stopper where such a compound is optionally laminated with one or more low processing temperature elastomeric, polymeric, and/or rubber lamination formulations (e.g., one or more suitable polyethylene such as a UHMWPE, copolymers thereof, and combinations thereof). In another instance, the second elastomeric, polymeric, and/or rubber formulation is formed from any one or more suitable elastomers, polymers, and/or rubbers having a processing temperature of at least 150° C., or at least 175° C., or even at least 200° C.

According to one aspect, the present invention is directed to two-part stopper comprising: a stopper cap formed from a first polymer material formed from a first elastomeric, polymeric, and/or rubber formulation that can be processed at a low temperature of less than 130° C.; and a stopper body formed from a second elastomeric, polymeric, and/or rubber formulation that can be processed at a higher temperature of more than 150° C., or more than 175° C.

In one embodiment, the first elastomeric, polymeric, and/or rubber formulation (hereinafter just “first polymer material”) comprises: at least one polymer compound having a Young's modulus in the range of about 5 MPa to about 15 MPa and a Shore A hardness in the range of about 40 to about 120, wherein the first polymer material is processed at a temperature in the range of about 90° C. to about 130° C. In one embodiment, the processing of the first polymer material is defined as an increase in ultimate torque response as measured by cure rheometer (ASTM 05289) of at least 400 percent within 10 minutes of isothermal conditioning at a platen temperature between about 90° C. and about 130° C. In another embodiment, the first polymer material of the present invention is formed from a polymer compound having a Young's modulus in the range of about 7.5 MPa to about 12.5 MPa and a Shore A hardness in the range of about 40 to about 120. In still another embodiment, the first polymer material of the present invention is selected from one or more ethylene propylene polymers such as one or more ethylene propylene diene monomer rubbers (EPDMs). In still another embodiment, the first polymer material of the present invention is selected from one or more one or more styrene terpolymers. In still another embodiment, the one or more styrene terpolymers are selected from one or more crosslinked backbone unsaturated styrene terpolymers. In still another embodiment, the one or more styrene terpolymers are selected from at least one brominated isobutylene paramethyl-styrene terpolymers (BIMSM) rubber with no unsaturation on the backbone of the polymer. In still another embodiment, the first polymer material of the present invention further comprises from about 50 phr to about 100 phr (parts per hundred rubber) of at least one non-staining halogenated isobutylene-isoprene copolymer and/or brominated isobutylene paramethyl-styrene terpolymers (BIMSM) of low to high Mooney viscosity. In still another embodiment, the first polymer material of the present invention further comprises about 50 phr to about 70 phr of calcined magnesium silicate. In still another embodiment, the first polymer material of the present invention further comprises about 10 phr to about 150 phr of at least one silica filler. In still another embodiment, the at least one silica filler is modified with at least one coupling agent. In still another embodiment, the first polymer material of the present invention further comprises up to about 50 phr of at least one processing oil. In still another embodiment, the stopper cap formed from the first polymer material of the present invention further comprises at least one lamination layer formed from one or more low processing temperature elastomeric, polymeric, and/or rubber lamination formulations (e.g., one or more suitable polyethylene such as a UHMWPE, copolymers thereof, and combinations thereof).

In another embodiment, the present invention is directed to a two-part stopper comprising: a stopper cap formed from any of the first elastomeric, polymeric, and/or rubber materials disclosed herein; a stopper body formed from any of the second elastomeric, polymeric, and/or rubber materials disclosed herein; and at least one sealing rib formed on at least one portion of the stopper cap and/or stopper body. In still another embodiment, the stopper cap further comprises a first portion of a sealing rib formed thereon and the adjacent portion of the stopper body has the second, or remainder, portion of the same sealing rib formed thereon, such that when the complete two-part stopper is assembled the first and second portions form a complete scaling rib.

In still another embodiment, the stopper cap further comprises at least one sealing rib formed thereon. In still another embodiment, the stopper body further comprises at least two scaling ribs formed thereon. In still another embodiment, the stopper cap has one sealing rib formed thereon and the stopper body has two or more sealing ribs formed thereon. In still another embodiment, the stopper cap is laminated with at least one lamination layer. In still another embodiment, all of the sealing ribs are laminated with at least one lamination layer. In still another embodiment, all of the sealing ribs and the stopper body are laminated with at least one lamination layer. In still another embodiment, the least one lamination layer is formed from at least one compound selected form a polyethylene such as a UHMWPE, copolymers thereof, and combinations thereof. In still another embodiment, the at least one lamination layer is formed from at least one layer of UHMWPE.

In still another embodiment, the present invention is directed to a syringe comprising: a syringe barrel; a plunger, wherein the plunger is designed to fit within the syringe barrel; and a two-part stopper according to any of the embodiments disclosed herein and formed from any of the elastomeric, polymeric and/or rubber materials disclosed herein, wherein the two-part stopper is adapted for attachment to one end of the plunger so as act as a seal for the syringe barrel.

In still another embodiment, the present invention is directed to a two-part sealing device comprising: a sealing cap formed from any of the first elastomeric, polymeric, and/or rubber materials disclosed herein; a sealing body formed from any of the second elastomeric, polymeric, and/or rubber materials disclosed herein; and at least one sealing structure and/or or sealing rib formed on at least one portion of either one or both of the sealing cap and/or the sealing body, wherein such a two-part sealing device can be utilized to seal any type of medical device and/or other device needing an adequate scaling device.

Clause 1: A two-part stopper comprising: a stopper cap formed from a first polymer material formed from a first polymer material that can be processed at a low temperature of less than 130° C.; and a stopper body formed from a second polymer material that can be processed at a higher temperature of more than 150° C.

Clause 2: The two-part stopper of clause 1, wherein the processing of the first polymer material is defined as an increase in ultimate torque response as measured by cure rheometer (ASTM 05289) of at least 400 percent within 10 minutes of isothermal conditioning at a platen temperature between about 90° C. and about 130° C.

Clause 3: The two-part stopper of any of clauses 1 or 2, wherein first polymer material has a Young's modulus in the range of about 7.5 MPa to about 12.5 MPa and a Shore A hardness in the range of about 40 to about 120.

Clause 4: The two-part stopper of any of clauses 1 to 3, wherein the first polymer material is selected from one or more ethylene propylene-containing polymers such as one or more ethylene propylene diene monomer rubbers (EPDMs) or from one or more styrene terpolymers, and optionally, the one or more styrene terpolymers are selected from one or more crosslinked backbone unsaturated styrene terpolymers, and further optionally, the one or more styrene terpolymers are selected from at least one brominated isobutylene paramethyl-styrene terpolymers (BIMSM) rubber with no unsaturation on the backbone of the polymer.

Clause 5: The two-part stopper of any of clauses 1 to 4, wherein either one, or both, of the first polymer material and/or the second polymer material further comprises one or more additives, one or more fillers, one or more processing aids, one or more oils, one or more dyes or colorants, or combinations of two or more thereof.

Clause 6: The two-part stopper of any of clauses 1 to 5, wherein at least one of the stopper cap and/or the stopper body is at least partially laminated.

Clause 7: The two-part stopper of any of clauses 1 to 5, wherein the stopper cap is laminated.

Clause 8: The two-part stopper of any of clauses 1 to 7, wherein at least one of the stopper cap and/or the stopper body further comprises at least one sealing rib formed thereon.

Clause 9: The two-part stopper of clause 8, wherein the stopper cap has a sealing rib formed thereon.

Clause 10: The two-part stopper of clause 9, wherein the stopper body has at least two scaling ribs formed thereon.

Clause 11: A syringe comprising: a syringe barrel; a plunger, wherein the plunger is designed to fit within the syringe barrel; and a two-part stopper according to any of clauses 1 to 10, wherein the stopper is adapted for attachment to one end of the plunger so as act as a seal for the syringe barrel.

Clause 12: A method for making a two-part stopper, the method comprising the steps of: supplying a first polymer material, wherein the first polymer material can be processed at a low temperature of less than 130° C.; forming a stopper cap from the first polymer material; supplying a second polymer material, wherein the first polymer material can be processed at a higher temperature of more than 150° C.; and forming a stopper body from the second polymer material, wherein the two-part stopper is formed by a co-injection molding process or a sequential injection molding process.

Clause 13: The method of clause 12, wherein the processing of the first polymer material is defined as an increase in ultimate torque response as measured by cure rheometer (ASTM 05289) of at least 400 percent within 10 minutes of isothermal conditioning at a platen temperature between about 90° C. and about 130° C.

Clause 14: The method of any of clauses 12 or 13, wherein first polymer material has a Young's modulus in the range of about 7.5 MPa to about 12.5 MPa and a Shore A hardness in the range of about 40 to about 120.

Clause 15: The method of any of clauses 12 to 14, wherein the first polymer material is selected from one or more ethylene propylene-containing polymers such as one or more ethylene propylene diene monomer rubbers (EPDMs) or from one or more styrene terpolymers, and optionally, the one or more styrene terpolymers are selected from one or more crosslinked backbone unsaturated styrene terpolymers, and further optionally, the one or more styrene terpolymers are selected from at least one brominated isobutylene paramethyl-styrene terpolymers (BIMSM) rubber with no unsaturation on the backbone of the polymer.

Clause 16: The method of any of clauses 12 to 15, wherein either one, or both, of the first polymer material and/or the second polymer material further comprises one or more additives, one or more fillers, one or more processing aids, one or more oils, one or more dyes or colorants, or combinations of two or more thereof.

Clause 17: The method of any of clauses 12 to 16, wherein at least one of the stopper cap and/or the stopper body is at least partially laminated.

Clause 18: The method of any of clauses 12 to 17, wherein the stopper cap is laminated.

Clause 19: The method of any of clauses 12 to 18, wherein at least one of the stopper cap and/or the stopper body further comprises at least one sealing rib formed thereon.

Clause 20: The method of clause 19, wherein the stopper cap has a sealing rib formed thereon.

Clause 21: The method of clause 19, wherein the stopper body has at least two sealing ribs formed thereon.

### BRIEF DESCRIPTION OF THE DRAWINGS

FIG. 1 is perspective view of an exemplary syringe with which embodiments of the disclosure may be implemented;

FIG. 2 is a perspective view of the syringe of FIG. 1 showing a needle shield covering a distal end of the syringe;

FIG. 3 is a perspective view of a stopper according to a first embodiment of the present invention;

FIG. 4 is a cross-sectional view of one stopper design according to one embodiment of the present invention where the stopper is fully laminated and the stopper contains an Acme thread therein; and

FIG. 5 is a cross-sectional view of one stopper design according to another embodiment of the present invention where the only the stopper cap of the stopper is laminated and the stopper contains an Acme thread therein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

As noted above, the invention relates, in general, to various elastomeric, polymeric, and/or rubber formulations that can be used to form two-part, or dual-formulation, stopper designs where at least a portion of the stopper is formed using a low temperature processing step while the remainder of the stopper is formed using a high temperature processing step, methods of making same, and uses for such stoppers. In one embodiment, the present invention relates to a two-part stopper design formed from a stopper cap (or the distal or front portion) formed from a first elastomeric, polymeric, and/or rubber formulation that can be processed at a low temperatures (e.g., less than 130° C.), and a stopper body formed from a second elastomeric, polymeric, and/or rubber formulation that can be processed at a higher temperature (e.g., more than 150° C., or more than 175° C.) than the processing temperature used to produce the stopper cap. In one instance, the first elastomeric, polymeric, and/or rubber formulation is formed from one or more suitable elastomers, polymers, and/or rubbers such as one or more ethylene propylene-containing polymers such as one or more ethylene propylene diene monomer rubbers (EPDMs), or one or more crosslinked backbone unsaturated styrene terpolymers and are used to form at least the stopper cap (or the distal or front portion) of a stopper where such a compound is optionally laminated with one or more low processing temperature elastomeric, polymeric, and/or rubber lamination formulations (e.g., one or more suitable polyethylene such as a UHMWPE, copolymers thereof, and combinations thereof). In another instance, the second elastomeric, polymeric, and/or rubber formulation is formed from any one or more suitable elastomers, polymers, and/or rubbers having a processing temperature of at least 150° C., or at least 175° C., or even at least 200° C.

Initially, a discussion of one set of exemplary stopper designs will be undertaken. However, it is to be understood that the following stopper design is only representative and the present invention is not limited to solely the designs disclosed herein. Rather, any stopper design where total sealing is desired can be utilized in conjunction with the present invention.

For purposes of the description hereinafter, the terms “upper”, “lower”, “right”, “left”, “vertical”, “horizontal”, “top”, “bottom”, “lateral”, “longitudinal”, and derivatives thereof, shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

As used herein, the terms “rubber composition”, “rubber material”, “compounded rubber”, “compounded rubber material”, “rubber compound”, “polymer composition”, “polymer material”, “compounded polymer”, “compounded polymer material”, and “polymer compound” are used interchangeably to refer to a polymer/rubber which has been blended or mixed with one or more various ingredients and/or materials as described herein.

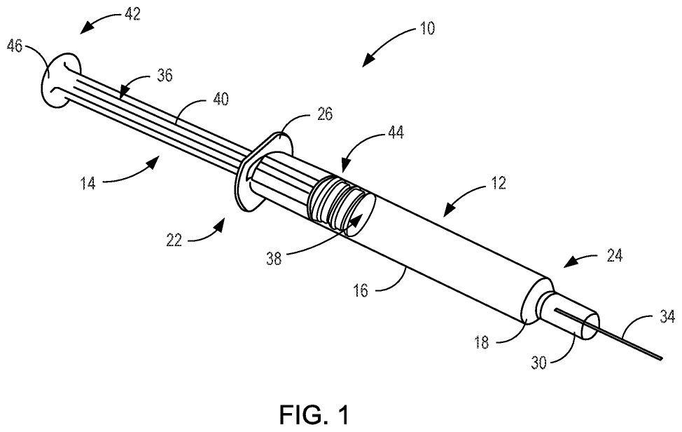

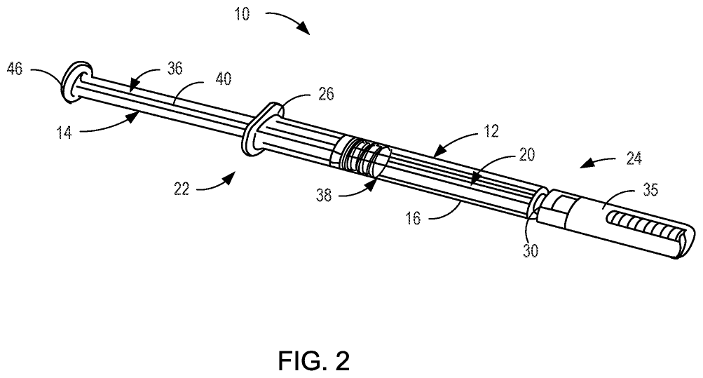

Referring to FIGS. 1 and 2, shown is a non-limiting embodiment of a syringe 10 with which aspects or embodiments of the disclosure may be implemented. According to some aspects of the disclosure, syringe 10 may be provided as a prefilled syringe, which provides the convenience of rapidly delivering the liquid therein to a patient without the need to first aspirate the medication from another container and meter its volume.

As shown in FIGS. 1 and 2, syringe 10 generally includes a syringe barrel 12 and a plunger assembly 14. Plunger assembly 14 is movable within syringe barrel 12 along a longitudinal axis to an advanced position to facilitate administering of an injectable fluid (e.g., medication) to a patient, for example. Syringe barrel 12 is formed from a suitable glass or plastic material and includes a generally cylindrical outer wall 16 and an end member 18 that collectively define a chamber 20 for retaining fluid therein. Syringe barrel 12 includes an open proximal end 22 configured to receive plunger assembly 14 therein and a distal end 24 at which end member 18 is positioned. Proximal end 22 of syringe barrel 12 may include a flange 26 to facilitate handling and positioning of syringe 10 and to maintain the relative position of syringe barrel 12 with respect to plunger assembly 14 during medication administration. At distal end 24, end member 18 may include a hub 30 which narrows with respect to cylindrical outer wall 16 and extends out distally therefrom. Hub portion 30 is formed as a partially hollow member that defines a channel therethrough in fluid communication with chamber 20. A needle 34 is attached to hub portion 30 within the channel, such as by being glued or otherwise secured to hub portion 30. According to some aspects of the disclosure, syringe 10 may further include a cover 35 that couples to hub portion 30 of syringe barrel 12 to protect needle 34.

Plunger assembly 14 of syringe 10 is formed of an elongate plunger rod 36 (more generally “plunger 36”, as used hereafter) and a two-part plunger head or stopper 38. Plunger 36 may include a main body 40 extending between a plunger proximal end 42 and a plunger distal end 44. A thumb press 46 is positioned at plunger proximal end 42 that may be engaged by a thumb (or other finger) of the user to apply a distally directed force to plunger assembly 14 to move plunger 36 with respect to syringe barrel 12. Plunger distal end 44 is configured to mate with two-part stopper 38. In other embodiments, plunger distal end 44 may be a threaded end that mates with two-part stopper 38.

Two-part stopper 38 of plunger assembly 14 is positioned at plunger distal end 44 so as to be movable along with plunger 36 within chamber 20 of syringe barrel 12. That is, as a distally-directed force is applied to plunger rod 36 (such as to thumb press 46), both plunger rod 36 and two-part stopper 38 are caused to move distally through syringe barrel 12, with two-part stopper 38 configured to maintain a seal with an inner surface of syringe barrel 12 as it is moved therethrough.

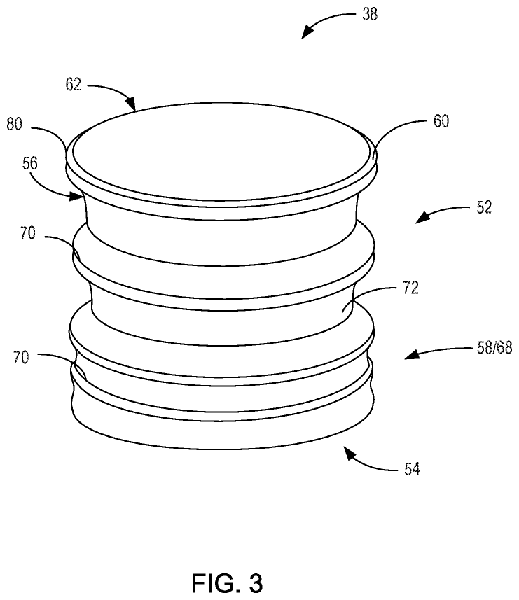

Referring now to FIG. 3, and with continued reference to FIGS. 1 and 2, the structure of an exemplary two-part stopper 38 that may be included in syringe 10 is shown in greater detail, according to a non-limiting aspect or embodiment of the disclosure. Two-part stopper 38 is defined by a main stopper body 52 that includes a proximal end 54 and a distal end 56. Proximal end 54 may be configured as an open proximal end that engages with the distal end of plunger 36, while distal end 56 is configured as a closed distal end configured to engage with and mate with stopper cap 60 so that stopper cap 60 is able to engage barrel 12 of syringe 10. Closed distal end 56 of main stopper body 52 includes a generally cylindrical portion 58 and is, in one non-limiting embodiment, formed to be integrally mated with a stopper cap portion 60. In another embodiment, main stopper body 52 and topper cap 60 can be formed independently and then assembled together to form two-part stopper 38 via an suitable bonding method including, but not limited, to an adhesive, polymer welding, ultrasonic welding, etc. In some embodiments, stopper cap portion 60 is configured as a flat surface to provide closed distal end 62, but it is recognized that stopper cap portion 60 may instead be configured as a conical or domed surface, as other non-limiting examples.

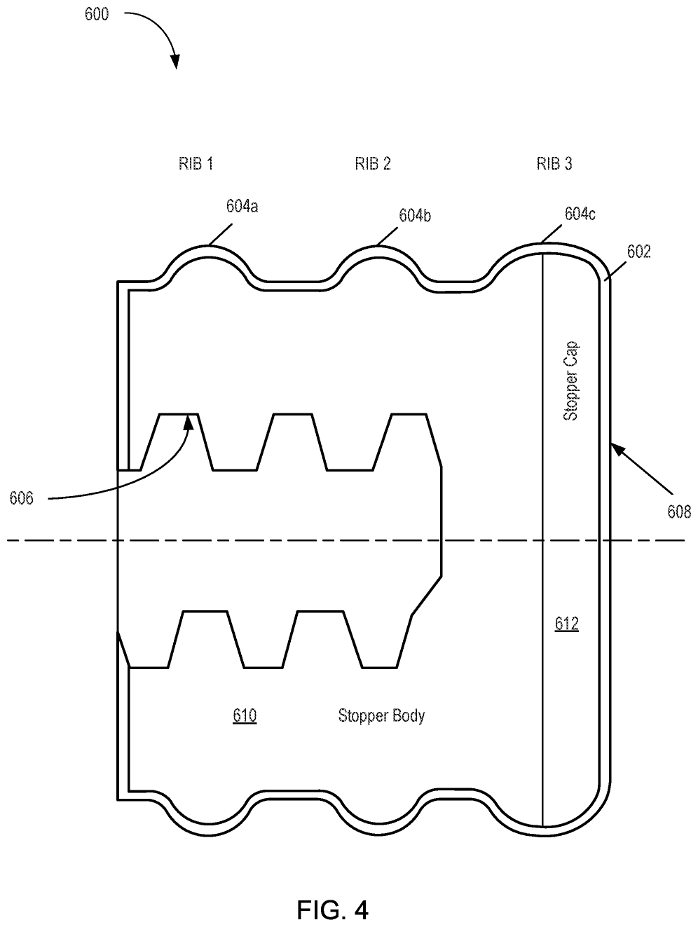

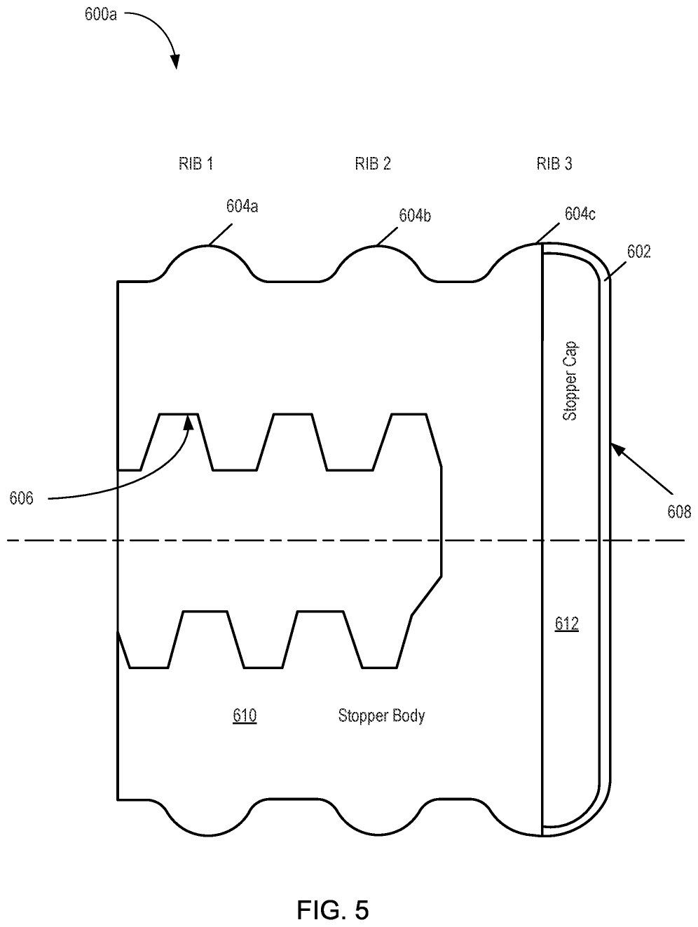

As known in the art, main stopper body 52 of two-part stopper 38 is partially hollow and is sized and configured to receive distal end 44 of plunger 36 therein. Main stopper body 52 of two-part stopper 38 defines an inner cavity (visible in FIGS. 4 and 5) that, in some embodiments, may include a threaded inner surface that is configured to receive and mate with a threaded distal end 44 of plunger 36. In other embodiments, the inner cavity may define an inner contoured surface having a notch therein that is configured to receive a flanged extension member of plunger 36.

Outer surface 68 of cylindrical portion 58 of main stopper body 52 includes one or more ribs thereon that extend circumferentially around the entire outer surface 68 of cylindrical portion 58. In each of the illustrated embodiments, a plurality of ribs 70 are included on two-part stopper 38, but it is recognized that other embodiments could include only a single rib 70. Ribs 70 may be formed integrally with main stopper body 52, such as via a molding process. The plurality of ribs 70 may include two ribs 70, with a first or distal rib positioned toward distal end 56 of main stopper body 52 and a second or proximal rib positioned toward proximal end 54 of main stopper body 52, or may include three or more ribs 70 spaced apart along cylindrical portion 58, with an inter-rib region 72 provided between each adjacent pair of ribs. Inter-rib region 72 of cylindrical portion 58 may have a generally flat outer surface 68 or a curved outer surface 68.

### Stopper Cap and First Polymer Material

At least stopper cap 60 of two-part stopper 38 is made from a material that is capable of forming a tight seal with the syringe barrel 12 as it is advanced therethrough. In some embodiments, stopper cap 60 of two-part stopper 38 is desirably manufactured from any suitable elastomeric, polymeric, and/or rubber (hereinafter referred to inclusively as solely a “first polymer material” or a “polymer material” for brevity's sake) material with a Young's modulus (also known as the tensile modulus, clastic modulus or traction modulus) in a range of about 5 MPa to about 15 MPa, or from about 5.25 MPa to about 14.75 MPa, or from about 5.5 MPa to about 14.5 MPa, or from about 5.75 MPa to about 14.25 MPa, or from about 6 MPa to about 14 MPa, or from about 6.25 MPa to about 13.75 MPa, or from about 6.5 MPa to about 13.5 MPa, or from about 6.75 MPa to about 13.25 MPa, or from about 7 MPa to about 13 MPa, or from about 7.25 MPa to about 12.75 MPa, or from about 7.5 MPa to about 12.5 MPa, or from about 7.75 MPa to about 12.25 MPa, or from about 8 MPa to about 12 MPa, or from about 8.25 MPa to about 11.75 MPa, or from about 8.5 MPa to about 11.5 MPa, or from about 8.75 MPa to about 11.25 MPa, or from about 9 MPa to about 11 MPa, or from about 9.25 MPa to about 10.75 MPa, or from about 9.5 MPa to about 10.5 MPa, or from about 9.75 MPa to about 10.25 MPa, or even about 10 MPa. In another embodiment, stopper cap 60 of two-part stopper 38 is desirably manufactured from any suitable polymer material, that is a first polymer material, with a Young's modulus (also known as the tensile modulus, clastic modulus or traction modulus) in a range of about 5 MPa to about 15 MPa, or from about 5.1 MPa to about 14.9 MPa, or from about 5.2 MPa to about 14.8 MPa, or from about 5.3 MPa to about 14.7 MPa, or from about 5.4 MPa to about 14.6 MPa, or from about 5.5 MPa to about 14.5 MPa, or from about 5.6 MPa to about 14.4 MPa, or from about 5.7 MPa to about 14.3 MPa, or from about 5.8 MPa to about 14.2 MPa, or from about 5.9 MPa to about 14.1 MPa, or from about 6 MPa to about 14 MPa, or from about 6.1 MPa to about 13.9 MPa, or from about 6.2 MPa to about 13.8 MPa, or from about 6.3 MPa to about 13.7 MPa, or from about 6.4 MPa to about 13.6 MPa, or from about 6.5 MPa to about 13.5 MPa, or from about 6.6 MPa to about 13.4 MPa, or from about 6.7 MPa to about 13.3 MPa, or from about 6.8 MPa to about 13.2 MPa, or from about 6.9 MPa to about 13.1 MPa, or from about 7 MPa to about 13 MPa, or from about 7.1 MPa to about 12.9 MPa, or from about 7.2 MPa to about 12.8 MPa, or from about 7.3 MPa to about 12.7 MPa, or from about 7.4 MPa to about 12.6 MPa, or from about 7.5 MPa to about 12.5 MPa, or from about 7.6 MPa to about 12.4 MPa, or from about 7.7 MPa to about 12.3 MPa, or from about 7.8 MPa to about 12.2 MPa, or from about 7.9 MPa to about 12.1 MPa, or from about 8 MPa to about 12 MPa, or from about 8.1 MPa to about 11.9 MPa, or from about 8.2 MPa to about 11.8 MPa, or from about 8.3 MPa to about 11.7 MPa, or from about 8.4 MPa to about 11.6 MPa, or from about 8.5 MPa to about 11.5 MPa, or from about 8.6 MPa to about 11.4 MPa, or from about 8.7 MPa to about 11.3 MPa, or from about 8.8 MPa to about 11.2 MPa, or from about 8.9 MPa to about 11.1 MPa, or from about 9 MPa to about 11 MPa, or from about 9.1 MPa to about 10.9 MPa, or from about 9.2 MPa to about 10.8 MPa, or from about 9.3 MPa to about 10.7 MPa, or from about 9.4 MPa to about 10.6 MPa, or from about 9.5 MPa to about 10.5 MPa, or from about 9.6 MPa to about 10.4 MPa, or from about 9.7 MPa to about 10.3 MPa, or from about 9.8 MPa to about 10.2 MPa, or from about 9.9 MPa to about 10.1 MPa, or even about 10 MPa. Here, as well as elsewhere in the specification and claims, individual numerical values can be combined to form additional, or even new/non-disclosed, numerical ranges.

The desired Shore A hardness of the afore-mentioned suitable first polymer material for stopper cap 60 of two-part stopper 38 of the present invention is between about 40 to about 120, or from about 45 to about 115, or from about 50 to about 110, or from about 55 to about 105, or from about 60 to about 100, or from about 65 to about 95, or from about 70 to about 90, or from about 75 to about 85, or even about 80. In another embodiment, stopper cap 60 of two-part stopper 38 is desirably manufactured from any suitable polymer material with a Shore A hardness of about 40 to about 120, or from about 42 to about 118, or from about 44 to about 116, or from about 46 to about 114, or from about 48 to about 112, or from about 50 to about 110, or from about 52 to about 108, or from about 54 to about 106, or from about 56 to about 104, or from about 58 to about 102, or from about 60 to about 100, or from about 62 to about 98, or from about 64 to about 96, or from about 66 to about 94, or from about 68 to about 92, or from about 70 to about 90, or from about 72 to about 88, or from about 74 to about 86, or from about 76 to about 84, or from about 78 to about 82, or even about 80. Here, as well as elsewhere in the specification and claims, individual numerical values can be combined to form additional, or even new/non-disclosed, numerical ranges.

Suitable stopper cap polymer materials (i.e., the first polymer material) include, but are not limited to, one or more polyolefins (e.g., PE, PP, and their copolymers), one or more polyamides (e.g., nylons), one or more polyesters (e.g., PET), one or more polystyrenes, one or more polyurethanes, one or more polycarbonates, one or more acrylonitrile-butadiene-styrenes, one or more polyacrylates, one or more elastomeric rubbers, one or more styrene terpolymers, or blends of any two or more thereof that meet at least one of the Young's modulus and/or Shore A properties defined above. In another embodiment, suitable stopper cap polymer materials include, but are not limited to, polymer materials selected from one or more polyisoprenes (including, but not limited to, natural rubber), one or more polyisobutylenes, one or more synthetic polyisoprenes, one or more polybutadienes (commonly referred to collectively as butadiene rubbers), one or more chloroprene rubbers (e.g., polychloroprene, neoprene, etc.), one or more butyl rubbers (i.e., copolymers of isobutene and isoprene), one or more halogenated butyl rubbers (e.g., chloro butyl rubber or bromo butyl rubber), one or more styrene-butadiene rubbers (i.e., copolymers of styrene and butadiene), one or more nitrile rubbers (i.e., copolymers of butadiene and acrylonitrile), one or more hydrogenated nitrile rubbers, natural rubber, one or more ethylene propylene rubbers, one or more ethylene propylene diene rubbers, one or more crosslinked backbone unsaturated styrene terpolymers, or blends of any two or more thereof that are able to be processed at a temperature less than or equal to about 130° C., less than or equal to a temperature of about 125° C., less than or equal to a temperature of about 120° C., less than or equal to a temperature of about 115° C., less than or equal to a temperature of about 110° C., less than or equal to a temperature of about 105° C., less than or equal to a temperature of about 100° C., less than or equal to a temperature of about 95° C., or even a temperature of about 90° C. Here, as well as elsewhere in the specification and claims, individual numerical values can be combined to form additional, or even new/non-disclosed, numerical ranges.

In another embodiment, suitable stopper cap first polymer materials include, but are not limited to, one or more butadiene rubbers, one or more halogenated butyl rubbers (e.g., chloro butyl rubber or bromo butyl rubber), one or more ethylene propylene-containing polymers (such as one or more ethylene propylene diene monomer rubbers (EPDMs)) one or more crosslinked backbone unsaturated styrene terpolymers, or blends of any two or more thereof.

In another embodiment, the first polymer/rubber material of the present invention can be any polymer/rubber material that can be processed using a process that is conducted at a temperature below about 130° C. In still another embodiment, the first polymer/rubber material of the present invention can be any polymer/rubber material that is partially, or fully, laminated with one or more lamination compounds disclosed herein that can be processed using a process that is conducted at a temperature below about 130° C. In still yet another embodiment, the processing processes disclosed herein are conducted at a temperature in the range of about 90° C. to about 130° C., or from about 91° C. to about 129° C., or from about 92° C. to about 128° C., or from about 93° C. to about 127° C., or from about 94° C. to about 126° C., or from about 95° C. to about 125° C., or from about 96° C. to about 124° C., or from about 97° C. to about 123° C., or from about 98° C. to about 122° C., or from about 99° C. to about 121° C., or from about 100° C. to about 120° C., or from about 101° C. to about 119° C., or from about 102° C. to about 118° C., or from about 103° C. to about 117° C., or from about 104° C. to about 116° C., or from about 105° C. to about 115° C., or from about 106° C. to about 114° C., or from about 107° C. to about 113° C., or from about 108° C. to about 112° C., or from about 109° C. to about 111° C., or even about 110° C. Here, as well as elsewhere in the specification and claims, individual numerical values can be combined to form additional, or even new/non-disclosed, numerical ranges. As used herein, the definition of processing completion is defined as an increase in ultimate torque response as measured by cure rheometer (ASTM 05289) of at least 400 percent within 10 minutes of isothermal conditioning at a platen temperature between about 90° C. and about 130° C. as further specified above. Thus, in one instance, the present invention involves a first polymer/rubber formulation/compound/material that involves the use of a specific processing method/system that is both fast and activated at a lower temperature in the range of about 90° C. and about 130° C. as further specified above.

In one embodiment, the present invention is directed to a first polymer/rubber composition comprising at least one brominated isobutylene paramethyl-styrene terpolymers (BIMSM) rubber with no unsaturation on the backbone of the polymer. Such polymers exhibit excellent barrier properties that make any stopper, or other closure or sealing device, ideal for a wide range of applications including, but not limited to, medical applications. As would be appreciated by those of skill in the art, the first polymer material of the present invention can further include one or more additives, one or more fillers, one or more processing aids, one or more oils, one or more dyes or colorants, etc., or combinations of two or more thereof as known in the art.

In another embodiment, stopper cap 60 of two-part stopper 38 is desirably manufactured from any suitable polymer material with either a Young's modulus and/or a Shore A hardness in one of the ranges noted above in combination with a high Mooney viscosity, where the base polymer/rubber material's Mooney viscosity is determined before curing. A high Mooney rubber is defined as rubber having Mooney viscosity greater than about 40, or greater than about 45, or greater than about 50, or greater than about 55, or greater than about 60, or greater than about 65, or even greater than about 70. Here, as well as elsewhere in the specification and claims, individual numerical values can be combined to form additional, or even new/non-disclosed, numerical ranges.

Suitable high Mooney viscosity rubbers include, but are not limited to, one or more polybutadiene rubbers, one or more polyisobutylene, one or more polyisoprene rubbers, natural rubber, one or more ethylene propylene rubbers, one or more ethylene propylene diene rubbers, one or more styrene-butadiene rubbers, or blends of any two or more thereof.

In one embodiment, stopper cap 60 of two-part stopper 38 is manufactured from a first polymer material with a significantly lower coefficient of friction and contact area/pressure relative to a conventional rubber stopper material. In one embodiment, the desired coefficient of friction of the afore-mentioned suitable first polymer materials for stopper cap 60 of two-part stopper 38 of the present invention is between about 0.1 to about 0.98, or from about 0.11 to about 0.97, or from about 0.12 to about 0.96, or from about 0.13 to about 0.95, or from about 0.14 to about 0.94, or from about 0.15 to about 0.93, or from about 0.16 to about 0.92, or from about 0.17 to about 0.91, or from about 0.18 to about 0.90, or from about 0.19 to about 0.89, or from about 0.2 to about 0.88, or from about 0.21 to about 0.87, or from about 0.22 to about 0.86, or from about 0.23 to about 0.85, or from about 0.24 to about 0.84, or from about 0.25 to about 0.83, or from about 0.26 to about 0.82, or from about 0.27 to about 0.81, or from about 0.28 to about 0.80, or from about 0.29 to about 0.79, or from about 0.3 to about 0.78, or from about 0.31 to about 0.77, or from about 0.32 to about 0.76, or from about 0.33 to about 0.75, or from about 0.34 to about 0.74, or from about 0.35 to about 0.73, or from about 0.36 to about 0.72, or from about 0.37 to about 0.71, or from about 0.38 to about 0.70, or from about 0.39 to about 0.69, or from about 0.4 to about 0.68, or from about 0.41 to about 0.67, or from about 0.42 to about 0.66, or from about 0.43 to about 0.65, or from about 0.44 to about 0.64, or from about 0.45 to about 0.63, or from about 0.46 to about 0.62, or from about 0.47 to about 0.61, or from about 0.48 to about 0.60, or from about 0.49 to about 0.59, or from about 0.5 to about 0.58, or from about 0.51 to about 0.57, or from about 0.52 to about 0.56, or from about 0.53 to about 0.55, or even about 0.54. Here, as well as elsewhere in the specification and claims, individual numerical values can be combined to form additional, or even new/non-disclosed, numerical ranges. Accordingly, in this embodiment, stopper 38 can be used with a syringe 10 without silicone oil or other lubricants. In addition, the syringe barrel 12 may be made of the same material, a similar material, or even a completely different material from that used to form either one or both portions 52 and 60 of stopper 38.

In one embodiment, the first polymer/rubber material that is used to form stopper cap 60 of the present invention has a rubber substrate modulus in a range of about 4 MPa to about 13 MPa, or from about 4.25 MPa to about 12.75 MPa, or from about 4.5 MPa to about 12.5 MPa, or from about 4.75 MPa to about 12.25 MPa, or from about 5 MPa to about 12 MPa, or from about 5.25 MPa to about 11.75 MPa, or from about 5.5 MPa to about 11.5 MPa, or from about 5.75 MPa to about 11.25 MPa, or from about 6 MPa to about 11 MPa, or from about 6.25 MPa to about 10.75 MPa, or from about 6.5 Mpa to about 10.5 Mpa, or from about 6.75 Mpa to about 10.25 MPa, or from about 7 MPa to about 10 MPa, or from about 7.25 MPa to about 9.75 MPa, or from about 7.5 MPa to about 9.5 MPa, or from about 7.75 MPa to about 9.25 MPa, or from about 8 MPa to about 9 MPa, or from about 8.25 MPa to about 8.75 MPa, or even about 8.5 MPa as measured at the limit of the stress versus strain of a molded part formed from a desired polymeric material without a laminated film thereon. Here, as well as elsewhere in the specification and claims, individual numerical values can be combined to form additional, or even new/non-disclosed, numerical ranges.

In another embodiment, the first polymer material that can be used to form stopper cap 60 in accordance with the present invention has a stress relaxation of between about 0 percent and about 35 percent as determined from initial contact max pressure. In still another embodiment, the first polymer material that is used to form stopper cap 60 of the present invention has a stress relaxation of between about 0 percent and about 30 percent, or between about 2.5 percent and about 27.5 percent, or between about 5 percent and about 25 percent, or between about 7.5 percent and about 22.5 percent, or between about 10 percent and about 20 percent, or between about 12.5 percent and about 17.5 percent, or even about 15 percent as determined from initial contact max pressure. Here, as well as elsewhere in the specification and claims, individual numerical values can be combined to form additional, or even new/non-disclosed, numerical ranges.

In one embodiment, the first polymer/rubber material for use in forming stopper cap 60 of the present invention is any crosslinked backbone unsaturated styrene terpolymer, or other polymer or rubber material, having a Shore A hardness in a range of about 40 to about 120, or from about 41 to about 119, or from about 42 to about 118, or from about 43 to about 117, or from about 44 to about 116, or from about 45 to about 115, or from about 46 to about 114, or from about 47 to about 113, or from about 48 to about 112, or from about 49 to about 111, or from about 50 to about 110, or from about 51 to about 109, or from about 52 to about 108, or from about 53 to about 107, or from about 54 to about 106, or from about 55 to about 105, or from about 56 to about 104, or from about 57 to about 103, or from about 58 to about 102, or from about 59 to about 101, or from about 60 to about 100, or from about 61 to about 99, or from about 62 to about 98, or from about 63 to about 97, or from about 64 to about 96, or from about 65 to about 95, or from about 66 to about 94, or from about 67 to about 93, or from about 68 to about 92, or from about 69 to about 91, or from about 70 to about 90, or from about 71 to about 89, or from about 72 to about 88, or from about 73 to about 87, or from about 74 to about 86, or from about 75 to about 85, or from about 76 to about 84, or from about 77 to about 83, or from about 78 to about 82, or from about 79 to about 81, or even about 80. Here, as well as elsewhere in the specification and claims, individual numerical values can be combined to form additional, or even new/non-disclosed, numerical ranges.

In another embodiment, the first polymer material for use in forming stopper cap 60 of the present invention is any crosslinked backbone unsaturated styrene terpolymer having a Young's modulus in a range of about 5 MPa to about 15 MPa, or from about 5.1 MPa to about 14.9 MPa, or from about 5.2 MPa to about 14.8 MPa, or from about 5.3 MPa to about 14.7 MPa, or from about 5.4 MPa to about 14.6 MPa, or from about 5.5 MPa to about 14.5 MPa, or from about 5.6 MPa to about 14.4 MPa, or from about 5.7 MPa to about 9.3 MPa, or from about 5.8 MPa to about 9.2 MPa, or from about 5.9 MPa to about 9.1 MPa, or from about 6 MPa to about 9 MPa, or from about 6.1 MPa to about 8.9 MPa, or from about 6.2 MPa to about 8.8 MPa, or from about 6.3 MPa to about 8.7 MPa, or from about 6.4 MPa to about 8.6 MPa, or from about 6.5 MPa to about 8.5 MPa, or from about 6.6 MPa to about 8.4 MPa, or from about 6.7 MPa to about 8.3 MPa, or from about 6.8 MPa to about 8.2 MPa, or from about 6.9 MPa to about 8.1 MPa, or from about 7 MPa to about 8 MPa, or from about 7.1 MPa to about 7.9 MPa, or from about 7.2 MPa to about 7.8 MPa, or from about 7.3 MPa to about 7.7 MPa, or from about 7.4 MPa to about 7.6 MPa, or even about 7.5 MPa. In still another embodiment, the first polymer material for use in forming stopper cap 60 of the present invention is any suitable butyl rubber material having a Shore A hardness within one of the above ranges in conjunction with a Young's modulus within one of the above ranges. Here, as well as elsewhere in the specification and claims, individual numerical values can be combined to form additional, or even new/non-disclosed, numerical ranges.

In another embodiment, the first polymer material for use in forming stopper cap 60 of the present invention is any suitable butyl rubber material having a stress relaxation over a period of 24 hours better than about 20 percent at room temperature (RT), better than about 25 percent at RT, or even better than about 30 percent at RT. In still another embodiment, the first polymer material for use in forming stopper cap 60 of the present invention is any of the polymeric materials disclosed above that have a stress relaxation better than about 21 percent at room temperature (RT), better than about 22 percent at RT, better than about 23 percent at RT, better than about 24 percent at RT, better than about 25 percent at RT, better than about 26 percent at RT, better than about 27 percent at RT, better than about 28 percent at RT, better than about 29 percent at RT, or even better than about 30 percent at RT. Here, as well as elsewhere in the specification and claims, individual numerical values can be combined to form additional, or even new/non-disclosed, numerical ranges.

In another embodiment, stopper cap 60 further comprises at least one sealing rib 80 formed thereon. In still another embodiment, stopper cap 60 is laminated with at least one lamination layer. In one instance, any optional lamination layer on stopper cap 60 is formed from at least one compound selected form a polyethylene such as a UHMWPE, copolymers thereof, and combinations thereof. In still another embodiment, any optional lamination layer on stopper cap 60 is formed from at least one layer of UHMWPE.

In another embodiment, stopper cap 60 is laminated with one or more fluoropolymer layers. In one embodiment, the one or more fluoropolymer layers described herein may include a single layer of densified expanded polytetrafluoroethylene (ePTFE), or the one or more fluoropolymer layers may include a composite fluoropolymer film having a barrier layer and a porous layer, the barrier layer including densified ePTFE, polytetrafluoroethylene (PTFE), fluorinated ethylene propylene (FEP), polyethylene (e.g., UHMWPE), polypropylene (e.g., syndiotactic-PP), polyvinylidene fluoride, polyvinylfluoride, perfluoropropylevinylether, a perfluoroalkoxy polymer, polysulfone, and copolymers and combinations thereof. In still another embodiment, any high temperature material, such as a polymeric material, can be used to form the one or more lamination layers regardless of whether stopper 38 according to this embodiment is partially, or even fully, laminated. In still another embodiment, the stoppers of the present invention are laminated with one or more non-fluoropolymer materials. Suitable non-fluoropolymer materials for such lamination layers include, but are not limited to, UHMWPE, syndiotactic-polypropylene blends, and/or polysulfone.

As noted above, stopper cap 60 can further comprises a first portion of a sealing rib formed thereon and the adjacent portion of stopper body 52 has the second, or remainder, portion of the same sealing rib formed thereon, such that when the complete two-part stopper is assembled the first and second portions form a complete sealing rib (not shown).

### Stopper Body and Second Polymer Material

Stopper body 52 can be formed from any suitable second polymer material with a processing temperature greater than or equal to about 150° C., or even greater than or equal to about 175° C. In one embodiment, such second polymer materials include, but are not limited to, one or more polyolefins (e.g., PE, PP, and their copolymers), one or more polyamides (e.g., nylons), one or more polyesters (e.g., PET), one or more polystyrenes, one or more polyurethanes, one or more polycarbonates, one or more acrylonitrile-butadiene-styrenes, one or more polyacrylates, one or more elastomeric rubbers, one or more styrene terpolymers, or blends of any two or more thereof that can be processed at a temperature of at least 150° C., or even at least 175° C. In another embodiment, suitable stopper body polymer materials include, but are not limited to, polymer materials selected from one or more polyisoprenes (including, but not limited to, natural rubber), one or more polyisobutylenes, one or more synthetic polyisoprenes, one or more polybutadienes (commonly referred to collectively as butadiene rubbers), one or more chloroprene rubbers (e.g., polychloroprene, neoprene, etc.), one or more butyl rubbers (i.e., copolymers of isobutene and isoprene), one or more halogenated butyl rubbers (e.g., chloro butyl rubber or bromo butyl rubber), one or more styrene-butadiene rubbers (i.e., copolymers of styrene and butadiene), one or more nitrile rubbers (i.e., copolymers of butadiene and acrylonitrile), one or more hydrogenated nitrile rubbers, natural rubber, one or more ethylene propylene rubbers, one or more ethylene propylene diene rubbers, one or more crosslinked backbone unsaturated styrene terpolymers, or blends of any two or more thereof that are able to be processed at a temperature greater than or equal to about 150° C., greater than or equal to a temperature of about 155° C., greater than or equal to a temperature of about 160° C., greater than or equal to a temperature of about 165° C., greater than or equal to a temperature of about 170° C., greater than or equal to a temperature of about 175° C., greater than or equal to a temperature of about 180° C., greater than or equal to a temperature of about 185° C., greater than or equal to a temperature of about 190° C., greater than or equal to a temperature of about 195° C., greater than or equal to a temperature of about 200° C., greater than or equal to a temperature of about 205° C., greater than or equal to a temperature of about 210° C., greater than or equal to a temperature of about 215° C., greater than or equal to a temperature of about 220° C., or even a temperature of about 225° C. Here, as well as elsewhere in the specification and claims, individual numerical values can be combined to form additional, or even new/non-disclosed, numerical ranges.

In another embodiment, suitable stopper body 52 second polymer materials include, but are not limited to, any polymer material that is known in the art for use in stoppers, medical closures, etc. so long as such material has a processing temperature of at least 150° C., or at least 175° C., or even at least 200° C.

As noted above, in some instances, stopper body 52 further comprises at least two scaling ribs 70 formed thereon. In still another embodiment, stopper cap 60 has one sealing rib 80 formed thereon and stopper body 52 has two or more sealing ribs 70 formed thereon. In still another embodiment, stopper cap 60 is laminated with at least one lamination layer as detailed above. In still another embodiment, all of the sealing ribs 70, 80 are laminated with at least one lamination layer. In still another embodiment, all of the sealing ribs 70, 80 and stopper body 52 are laminated with at least one lamination layer. In still another embodiment, all of stopper 38 is laminated after the formation and completion of both stopper body 52 and stopper cap 60 with at least one lamination layer. In still another embodiment, any of the aforementioned lamination layers are formed from at least one compound selected form a polyethylene such as a UHMWPE, copolymers thereof, and combinations thereof. In still another embodiment, any of the aforementioned lamination layers are formed from at least one layer of UHMWPE.

In another embodiment, stopper body 52 is partially, or even fully, laminated with one or more fluoropolymer layers (even if that is after the formation and completion of both stopper body 52 and stopper cap 60 so as to form a completed stopper 38). In one embodiment, the one or more fluoropolymer layers described herein may include a single layer of densified expanded polytetrafluoroethylene (ePTFE), or the one or more fluoropolymer layers may include a composite fluoropolymer film having a barrier layer and a porous layer, the barrier layer including densified ePTFE, polytetrafluoroethylene (PTFE), fluorinated ethylene propylene (FEP), polyethylene (e.g., UHMWPE), polypropylene (e.g., syndiotactic-PP), polyvinylidene fluoride, polyvinylfluoride, perfluoropropylevinylether, a perfluoroalkoxy polymer, polysulfone, and copolymers and combinations thereof. In still another embodiment, any high temperature material, such as a polymeric material, can be used to form the one or more lamination layers regardless of whether stopper 38 according to this embodiment is partially, or even fully, laminated. In still another embodiment, the stoppers of the present invention are laminated with one or more non-fluoropolymer materials. Suitable non-fluoropolymer materials for such lamination layers include, but are not limited to, UHMWPE, syndiotactic-polypropylene blends, and/or polysulfone.

As would be appreciated by those of skill in the art, the first polymer material of the present invention can further include one or more additives, one or more fillers, one or more processing aids, one or more oils, one or more dyes or colorants, etc., or combinations of two or more thereof as known in the art.

### Method of Manufacture

With reference now to FIGS. 4 and 5, additional stopper 38 designs are shown in accordance with alternative embodiments of the present invention. Turning to FIG. 4, FIG. 4 discloses a stopper 600 having a laminated surface 602 thereon. As can be seen from FIG. 4, stopper 600 has three ribs 604a, 604b, and 604c. Although stopper 600 has been shown with three ribs, ribs 604a, 604b, and 604c, with rib 604c being partially on stopper body 610 and stopper cap 612, the present invention is not limited thereto. Rather, as noted herein, any suitable number of ribs can be formed in stopper 600 as desired by the design parameters under consideration for stopper 600. In one embodiment, stopper 600 has a flat front surface 608, but as would be appreciated by those of skill in the art could alternatively have any desired geometric design at the front surface thereof.

Continuing on further, stopper 600 further has a threaded portion 606, which in a non-limiting instance, can be formed in the shape of an Acme thread structure 606. As disclosed therein, stopper 600 can be any suitable size as determined by its length and width. As illustrated in FIG. 4, stopper 600 is comprised of a stopper cap 612 formed from a first polymer material as described above and a stopper body 610 formed from a second polymer material as described above.

As such, stopper 600 of FIG. 4 has, in one non-limiting embodiment, three ribs, an Acme thread, a flat front, and is fully laminated. However, as is discussed herein, modifications to the design of stopper 600 are fully within the scope of the present invention and can include one or more exemplary non-limiting modifications such as a different number of ribs, one or more differently-shaped ribs, one or more laminated ribs, one or more non-laminated ribs, a non-flat front surface, etc.

Turning to FIG. 5, this figure represents a stopper design 600a where Acme thread structure 606 of stopper 600 has been replaced by a snap-in plunger rod connector 606a. Although stopper 600a has been shown with three ribs, ribs 604a, 604b, and 604c, with rib 604c being partially on stopper body 610 and stopper cap 612, the present invention is not limited thereto. Rather, as noted herein, any suitable number of ribs can be formed in stopper 600a as desired by the design parameters under consideration for stopper 600a. As illustrated in FIG. 5, stopper 600 is comprised of a stopper cap 612 formed from a first polymer material as described above and a stopper body 610 formed from a second polymer material as described above.

In one embodiment, stopper 600a has a flat front surface 608, but as would be appreciated by those of skill in the art could alternatively have any desired geometric design at the front surface thereof. As such, stopper 600a of FIG. 5 has, in one non-limiting embodiment, three ribs, a snap-in plunger rod connector, a flat front, and is partially laminated on only stopper cap 612. However, as is discussed herein, modifications to the design of stopper 600a are fully within the scope of the present invention and can include one or more exemplary non-limiting modifications such as a different number of ribs, one or more laminated ribs, one or more non-laminated ribs, a non-flat front surface, etc.

In various alternative embodiments, Acme thread structure 606 or snap-in plunger rod connector 606a can be replaced by a buttress thread structure, or any threaded structure designed to permit a stopper according to the present invention to be connected to any desired medical device such as a syringe, etc.

In one embodiment, flat front 608 can be modified to be any geometric shape known in the art including, but not limited to, a conical-shaped front surface, a nipple-containing front surface, etc.

In still another embodiment, any of the lamination layers disclosed herein can independently be formed from at least one compound selected form densified expanded polytetrafluoroethylene (ePTFE), polytetrafluoroethylene (PTFE), fluorinated ethylene propylene (FEP), a polyethylene such as a UHMWPE, polypropylene, polyvinylidene fluoride, polyvinylfluoride, perfluoropropylevinylether, a perfluoroalkoxy polymer, and copolymers and combinations thereof. In still another embodiment, t any of the lamination layers disclosed herein can independently be formed from at least one layer of polytetrafluoroethylene (PTFE) or UHMWPE.

In one embodiment, a stopper in accordance with the present invention is manufactured using a two-part co-injection mold where stopper cap 60 and/or 612 is formed in a low temperature zone (i.e., less than about 130° C., etc. as defined above) and stopper body 52 or 610 is formed in a high temperature zone (i.e., above about 150° C., or above about 175° C., etc. as defined above). Alternatively, a stopper in accordance with the present invention can be formed via a sequential molding process by first molding stopper cap 60 and/or 612 and then molding, in a second mold, the remainder of a stopper in accordance with the present invention by placing the pre-molded stopper cap 60 and/or 612 into a second mold with at least one heat controllable platen to ensure that the previously molded stopper cap is not subjected to too high a temperature (i.e., in excess of 130° C.).

Given the above, in one embodiment the present invention relates to a two-part stopper comprising: a stopper cap formed from a first polymer material formed from a first polymer material that can be processed at a low temperature of less than 130° C.; and a stopper body formed from a second polymer material that can be processed at a higher temperature of more than 150° C., or more than 175° C., or even more than 200° C. In one embodiment, the two-part stopper of the present invention utilizes a first polymer material where the processing of the first polymer material is defined as an increase in ultimate torque response as measured by cure rheometer (ASTM 05289) of at least 400 percent within 10 minutes of isothermal conditioning at a platen temperature between about 90° C. and about 130° C.

In another embodiment, the two-part stopper of the present invention utilizes a first polymer material having a Young's modulus in the range of about 7.5 MPa to about 12.5 MPa and a Shore A hardness in the range of about 40 to about 120. In another embodiment, the two-part stopper of the present invention utilizes a first polymer material that is selected from one or more propylene-containing polymers (e.g., ethylene propylene diene monomer rubbers), or one or more styrene terpolymers, and optionally, the one or more styrene terpolymers are selected from one or more crosslinked backbone unsaturated styrene terpolymers, and further optionally, the one or more styrene terpolymers are selected from at least one brominated isobutylene paramethyl-styrene terpolymers (BIMSM) rubber with no unsaturation on the backbone of the polymer.

In another embodiment, cither one, or both, of the first polymer material and/or the second polymer material further comprises one or more additives, one or more fillers, one or more processing aids, one or more oils, one or more dyes or colorants, or combinations of two or more thereof. In another embodiment, at least one of the stopper cap and/or the stopper body is at least partially laminated. In another embodiment, the stopper cap is laminated. In another embodiment, at least one of the stopper cap and/or the stopper body further comprises at least one scaling rib formed thereon. In another embodiment, the stopper cap has a sealing rib formed thereon. In another embodiment, the stopper body has at least two sealing ribs formed thereon.

In still another embodiment, the present invention relates to a syringe comprising: a syringe barrel; a plunger, wherein the plunger is designed to fit within the syringe barrel; and a two-part stopper according to any of the embodiments disclosed herein, wherein the stopper is adapted for attachment to one end of the plunger so as act as a seal for the syringe barrel.

In still another embodiment, the present invention relates to method for making a two-part stopper, the method comprising the steps of: supplying a first polymer material, wherein the first polymer material can be processed at a low temperature of less than 130° C.; forming a stopper cap from the first polymer material; supplying a second polymer material, wherein the first polymer material can be processed at a higher temperature of more than 150° C., or more than 175° C.; and forming a stopper body from the second polymer material, wherein the two-part stopper is formed by a co-injection molding process or a sequential injection molding process.

In still another embodiment, the processing of the first polymer material used in a method according to the present invention is defined as an increase in ultimate torque response as measured by cure rheometer (ASTM 05289) of at least 400 percent within 10 minutes of isothermal conditioning at a platen temperature between about 90° C. and about 130° C. In still another embodiment, the first polymer material used in a method according to the present invention has a Young's modulus in the range of about 7.5 MPa to about 12.5 MPa and a Shore A hardness in the range of about 40 to about 120.

In still another embodiment, the first polymer material used in a method according to the present invention is selected from one or more styrene terpolymers, and optionally, the one or more styrene terpolymers are selected from one or more propylene-containing polymers (e.g., ethylene propylene diene monomer rubbers), or one or more crosslinked backbone unsaturated styrene terpolymers, and further optionally, the one or more styrene terpolymers are selected from at least one brominated isobutylene paramethyl-styrene terpolymers (BIMSM) rubber with no unsaturation on the backbone of the polymer. In still another embodiment, either one, or both, of the first polymer material and/or the second polymer material utilized used in a method according to the present invention further comprises one or more additives, one or more fillers, one or more processing aids, one or more oils, one or more dyes or colorants, or combinations of two or more thereof.

In still another embodiment, a stopper formed in accordance with a method of the present invention has at least one of the stopper cap and/or the stopper body that is at least partially laminated. In still another embodiment, the stopper cap formed by a method according to the present invention is laminated. In still another embodiment, at least one of the stopper cap and/or the stopper body of any stopper formed in accordance with a method of the present invention further comprises at least one sealing rib formed thereon. In still another embodiment, the stopper cap formed in accordance with the present invention has a sealing rib formed thereon. In still another embodiment, the stopper body formed in accordance with the present invention has at least two sealing ribs formed thereon.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure that are known or customarily practiced in the art to which this disclosure pertains and which fall within the limits of the appended claims.

What is claimed is:

1. A two-part stopper comprising:
a stopper cap formed from a first polymer material formed from a first polymer material that can be processed at a low temperature of less than 130° C.; and
a stopper body formed from a second polymer material that can be processed at a higher temperature of more than 150° C.

2. The two-part stopper of claim 1, wherein the processing of the first polymer material is defined as an increase in ultimate torque response as measured by cure rheometer (ASTM 05289) of at least 400 percent within 10 minutes of isothermal conditioning at a platen temperature between about 90° C. and about 130° C.

3. The two-part stopper of claim 1, wherein first polymer material has a Young's modulus in the range of about 7.5 MPa to about 12.5 MPa and a Shore A hardness in the range of about 40 to about 120.

4. The two-part stopper of claim 1, wherein the first polymer material is selected from one or more propylene-containing polymers or one or more styrene terpolymers, and optionally, the one or more styrene terpolymers are selected from one or more crosslinked backbone unsaturated styrene terpolymers, and further optionally, the one or more styrene terpolymers are selected from at least one brominated isobutylene paramethyl-styrene terpolymers (BIMSM) rubber with no unsaturation on the backbone of the polymer.

5. The two-part stopper of claim 1, wherein either one, or both, of the first polymer material and/or the second polymer material further comprises one or more additives, one or more fillers, one or more processing aids, one or more oils, one or more dyes or colorants, or combinations of two or more thereof.

6. The two-part stopper of claim 1, wherein at least one of the stopper cap and/or the stopper body is at least partially laminated.

7. The two-part stopper of claim 1, wherein the stopper cap is laminated.

8. The two-part stopper of claim 1, wherein at least one of the stopper cap and/or the stopper body further comprises at least one sealing rib formed thereon.

9. The two-part stopper of claim 8, wherein the stopper cap has a sealing rib formed thereon.

10. The two-part stopper of claim 9, wherein the stopper body has at least two sealing ribs formed thereon.

11. A syringe comprising:
a syringe barrel;
a plunger, wherein the plunger is designed to fit within the syringe barrel; and
a two-part stopper having a stopper cap formed from a first polymer material formed from a first polymer material that can be processed at a low temperature of less than 130° C., and a stopper body formed from a second polymer material that can be processed at a higher temperature of more than 150° C., wherein the stopper is adapted for attachment to one end of the plunger so as act as a seal for the syringe barrel.

12. A method for making a two-part stopper, the method comprising the steps of:
supplying a first polymer material, wherein the first polymer material can be processed at a low temperature of less than 130° C.;
forming a stopper cap from the first polymer material;
supplying a second polymer material, wherein the first polymer material can be processed at a higher temperature of more than 150° C.; and
forming a stopper body from the second polymer material,
wherein the two-part stopper is formed by a co-injection molding process or a sequential injection molding process.

13. The method of claim 12, wherein the processing of the first polymer material is defined as an increase in ultimate torque response as measured by cure rheometer (ASTM 05289) of at least 400 percent within 10 minutes of isothermal conditioning at a platen temperature between about 90° C. and about 130° C.

14. The method of claim 12, wherein first polymer material has a Young's modulus in the range of about 7.5 MPa to about 12.5 MPa and a Shore A hardness in the range of about 40 to about 120.

15. The method of claim 12, wherein the first polymer material is selected from one or more propylene-containing polymers or one or more styrene terpolymers, and optionally, the one or more styrene terpolymers are selected from one or more crosslinked backbone unsaturated styrene terpolymers, and further optionally, the one or more styrene terpolymers are selected from at least one brominated isobutylene paramethyl-styrene terpolymers (BIMSM) rubber with no unsaturation on the backbone of the polymer.

16. The method of claim 12, wherein either one, or both, of the first polymer material and/or the second polymer material further comprises one or more additives, one or more fillers, one or more processing aids, one or more oils, one or more dyes or colorants, or combinations of two or more thereof.

17. The method of claim 12, wherein at least one of the stopper cap and/or the stopper body is at least partially laminated.

18. The method of claim 12, wherein the stopper cap is laminated.

19. The method of claim 12, wherein at least one of the stopper cap and/or the stopper body further comprises at least one sealing rib formed thereon.

20. The method of claim 19, wherein the stopper cap has a scaling rib formed thereon.
